(19) United States
(12) Patent Application Publication
(10) Pub. No.: US 2009/0042773 A1
(43) Pub. Date: Feb. 12, 2009

(54) P13 kinase gamma inhibitors for the treatment of anaemia

(76) Inventors: Reinhard Wetzker, Jena (DE); Angelika Mueller, Jena (DE); Christian Rommel, Geneva (CH)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/664,969

(22) PCT Filed: Oct. 11, 2005
(86) PCT No.: PCT/EP05/55156

§ 371 Date: Jul. 10, 2007

(30) Foreign Application Priority Data
Oct. 12, 2004 (EP) 04104997.4

(51) Int. Cl. A61K 38/00; A61K 31/536; A61K 31/498; A61K 31/517; A61K 31/4709; A61K 31/427

(52) U.S. Cl. 514/8; 514/230.5; 514/249; 514/266.2; 514/314; 514/369

(57) ABSTRACT

This present invention is related to the use of selective PD Kinase gamma inhibitors for the manufacture of a medicament for the treatment of disorders related to erythrocyte deficiency. Specifically, the present invention is related to the use of selective PI3 Kinase gamma inhibitors, e.g. substituted azolidinone-vinyl fused-benzene derivatives for the treatment of an anaemia, including haemolytic anaemia, aplastic anaemia and pure red cell anaemia. (I) wherein A, X, Y₁, Y₂, Z, n, R¹ and R² are described in details in the description hereinafter.

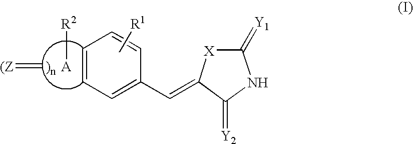

### FIELD OF THE INVENTION

This present invention is related to the use of selective PI3 Kinase gamma inhibitors for the manufacture of a medicament for the treatment of disorders related to erythrocyte deficiency. Specifically, the present invention is related to the use of selective PI3 Kinase gamma inhibitors, e.g. substituted azolidinone-vinyl fused-benzene derivatives for the treatment of an anaemia, including haemolytic anaemia, aplastic anaemia and pure red cell anaemia.

### BACKGROUND OF THE INVENTION

Erythropoietin for use in the treatment of anaemia:

Erythropoietin (EPO) is a glycoprotein and is the primary regulator of the proliferation and differentiation of immature erythroid cells (erythrogenesis). EPO is produced in the fetal liver and in the adult kidney in response to hypoxia (low oxygen levels in blood or tissue). It circulates in the blood stream where it targets the EPO receptor (EPOR) on committed progenitor cells in the bone marrow and other hematopoietic tissues. Recombinant human erythropoietin (rHuEPO) is widely used in therapy of patients with anaemia due to chronic renal failure, cancer chemotherapy and AZT treatment. Recombinant human erythropoietin (rHuEpo or epoetin alfa) is commercially available as EPOGEN® (epoetin alfa, recombinant human erythropoietin) (Amgen Inc., Thousand Oaks, Calif.); Recormon (Roche) and as PROCRIT® (epoetin alfa, recombinant human erythropoietin) (Ortho Biotech Inc., Raritan, N.J.).

When used therapeutically, EPO is administered either by intravenous or subcutaneous injection. The administered dosage of EPO usually does not exceed 720 IU/kg of body weight.

The fact that EPO is a relatively large glycoprotein adversely impacts the cost of manufacture and the mode of delivery of this therapeutic agent.

Given the importance of erythropoietin, it would be very desirable to be able to identify organic molecules capable of replacing EPO or at least to strengthen or boost the effect normally elicited by EPO.

PI3 Kinases:

Cellular plasma membranes can be viewed as a large store of second messengers that can be enlisted in a variety of signal transduction pathways. As regards function and regulation of effector enzymes in phospholipid signalling pathways, these enzymes generate second messengers from the membrane phospholipid pool (class I PI3 kinases (e.g. PI3K gamma)) are dual-specific kinase enzymes, means they display both: lipid kinase (phosphorylation of phospho-inositides) as well as protein kinase activity, shown to be capable of phosphorylation of other protein substrates, including auto-phosphorylation as intra-molecular regulatory mechanism. These enzymes of phospholipid signalling are activated in response to a variety of extra-cellular signals such as growth factors, mitogens, integrins (cell-cell interactions) hormones, cytokines, viruses and neurotransmitters such as described in Scheme 1 hereinafter and also by intra-cellular cross regulation by other signaling molecules (cross-talk where the original signal can activate some parallel pathways that in a second step transmit signals to PI3Ks by intra-cellular signaling events), such as small GTPases, kinases or phosphatases for example.

The inositol phospholipids (phosphoinositides) intracellular signalling pathway begins with binding of a signalling molecule (extracellular ligands, stimuli, receptor dimerization, transactivation by heterologous receptor (e.g. receptor tyrosine kinase)) to a G-protein linked transmembrane receptor integrated into the plasma membrane.

PI3K converts the membrane phospholipid PIP(4,5)2 into PIP(3,4,5)3 which in turn can be further converted into another 3′ phosphorylated form of phosphoinositides by 5′-specific phospho-inositide phosphatases, thus PI3K enzymatic activity results either directly or indirectly in the generation of two 3′-phosphoinositide subtypes that function as 2^{nd} messengers in intra-cellular signal transduction (Trends Biochem Sci. 22(7) p. 267-72 (1997) by Vanhaesebroeck B et al., Chem. Rev. 101(8) p. 2365-80 (2001) by Leslie N. R et al (2001); Annu Rev Cell Dev Biol. 17 p. 615-75 (2001) by Katso R. et al. and Cell Mol Life Sci. 59(5) p. 761-79 (2002) by Toker a et al.). Multiple PI3K isoforms categorized by their catalytic subunits, their regulation by corresponding regulatory subunits, expression patterns and signaling-specific functions (p110α, β, δ, and γ) perform this enzymatic reaction (Exp Cell Res. 25(1) p. 239-54 (1999) by Vanhaesebroeck B. and Annu Rev Cell Dev Biol. 17 p. 615-75 (2001) by Katso R. et al).

The evolutionary conserved isoforms p110α and β are ubiquitiously expressed, while δ and γ are more specifically expressed in the haematopoetic cell system, smooth muscle cells, myocytes and endothelial cells (Trends Biochem Sci. 22(7) p. 267-72 (1997) by Vanhaesebroeck B et al.). Their expression might also be regulated in an inducible manner depending on the cellular-, tissue type and stimuli as well as disease context.

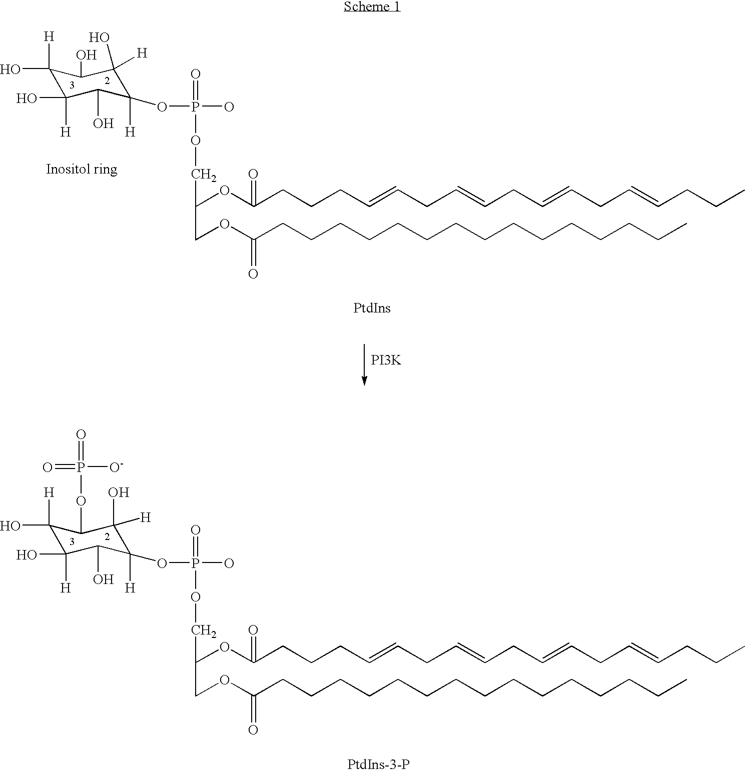

As above illustrated in Scheme 1, Phosphoinositide 3-kinase (PI3K) is involved in the phosphorylation of Phosphatidylinositol (PtdIns) on the third carbon of the inositol ring. The phosphorylation of PtdIns to 3,4,5-triphosphate (PtdIns(3,4,5)P₃), PtdIns(3,4)P₂ and PtdIns(3)P act as second messengers for a variety of signal transduction pathways, including those essential to cell proliferation, cell differentiation, cell growth, cell size, cell survival, apoptosis, adhesion, cell motility, cell migration, chemotaxis, invasion, cytoskeletal rearrangement, cell shape changes, vesicle trafficking and metabolic pathway.

Two compounds, LY294002 and wortmannin are known PI3-kinase inhibitors. These compounds are non-selective PI3K inhibitors

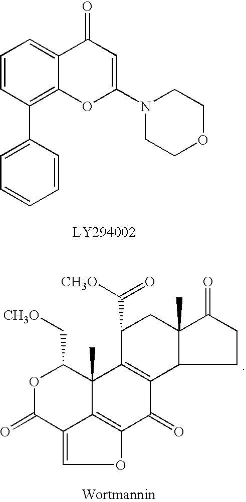

LY294002 has been reported to inhibit EPO induced erythropoiesis from CD34+ progenitor cells (June H. Myklebust et al., Experimental Hematology 30 (2002), 990). Also, it has been reported that Wortmannin prevents K562 erythroleukemia cells from EPO induced erythroid differentiation (L. Neri et al. Cellular Signalling 14 (2002) 21).

Azolidinone-vinyl benzene derivatives are described in PCT/EP02/100798. The compounds are said to be PI3 Kinase inhibitors, in particular of PI3 Kinase gamma and are said to be useful in the treatment and/or prophylaxis of autoimmune disorders and/or inflammatory diseases, cardiovascular diseases, neurodegenerative diseases, bacterial or viral infections, kidney diseases, platelet aggregation, cancer, graft rejection or lung injuries.

### SUMMARY OF THE INVENTION

It has now been surprisingly found that selective PI3 Kinase gamma inhibitors are useful for the treatment of disorders related to erythrocyte deficiency. Specifically, the present invention is related to the use of selective PD Kinase gamma inhibitors, e.g. substituted azolidinone-vinyl fused-benzene derivatives of formula (I) for the treatment of anemia, including haemolytic anaemia, aplastic anaemia, pure red cell anaemia.

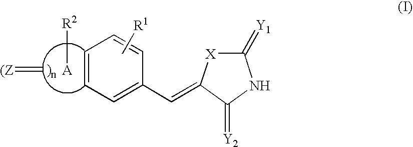

wherein A, X, Y₁, Y₂, Z, n, R¹ and R² are described in details in the description hereinafter.

### DESCRIPTION OF THE INVENTION

The following paragraphs provide definitions of the various chemical moieties that make up the compounds according to the invention and are intended to apply uniformly throughout the specification and claims unless an otherwise expressly set out definition provides a broader definition.

“C₁-C₆-alkyl” refers to monovalent alkyl groups having 1 to 6 carbon atoms. This term is exemplified by groups such as methyl, ethyl, n-propyl, isopropyl, n-butyl, isobutyl, tert-butyl, n-pentyl, n-hexyl and the like.

“Aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenantrenyl and the like.

“C₁-C₆-alkyl aryl” refers to C₁-C₆-alkyl groups having an aryl substituent, including benzyl, phenethyl and the like.

“Heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl.

“C₁-C₆-alkyl heteroaryl” refers to C₁-C₆-alkyl groups having a heteroaryl substituent, including 2-furylmethyl, 2-thienylmethyl, 2-(1H-indol-3-yl)ethyl and the like.

“C₂-C₆-alkenyl” refers to alkenyl groups preferably having from 2 to 6 carbon atoms and having at least 1 or 2 sites of alkenyl unsaturation. Preferable alkenyl groups include ethenyl (—CH═CH₂), n-2-propenyl (allyl, —CH₂CH═CH₂) and the like.

“C₂-C₆-alkenyl aryl” refers to C₂-C₆-alkenyl groups having an aryl substituent, including 2-phenylvinyl and the like.

“C₂-C₆-alkenyl heteroaryl” refers to C₂-C₆-alkenyl groups having a heteroaryl substituent, including 2-(3-pyridinyl)vinyl and the like.

“C₂-C₆-alkynyl” refers to alkynyl groups preferably having from 2 to 6 carbon atoms and having at least 1-2 sites of alkynyl unsaturation, preferred alkynyl groups include ethynyl (—C≡CH), propargyl (—CH₂C≡CH), and the like.

“C₂-C₆-alkynyl aryl” refers to C₂-C₆-alkynyl groups having an aryl substituent, including phenylethynyl and the like.

“C₂-C₆-alkynyl heteroaryl” refers to C₂-C₆-alkynyl groups having a heteroaryl substituent, including 2-thienylethynyl and the like.

“C₃-C₈-cycloalkyl” refers to a saturated carbocyclic group of from 3 to 8 carbon atoms having a single ring (e.g., cyclohexyl) or multiple condensed rings (e.g., norbornyl). Preferred cycloalkyl include cyclopentyl, cyclohexyl, norbornyl and the like.

“Heterocycloalkyl” refers to a C₃-C₈-cycloalkyl group according to the definition above, in which up to 3 carbon atoms are replaced by heteroatoms chosen from the group consisting of O, S, NR, R being defined as hydrogen or methyl. Preferred heterocycloalkyl include pyrrolidine, piperidine, piperazine, 1-methylpiperazine, morpholine, and the like.

“C₁-C₆-alkyl cycloalkyl” refers to C₁-C₆-alkyl groups having a cycloalkyl substituent, including cyclohexylmethyl, cyclopentylpropyl, and the like.

“C₁-C₆-alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

“Carboxy” refers to the group —(O)OH.

“C₁-C₆-alkyl carboxy” refers to C₁-C₆-alkyl groups having an carboxy substituent, including 2-carboxyethyl and the like.

“Acyl” refers to the group —C(O)R where R includes “C₁-C₆-alkyl”, “aryl”, “heteroaryl”, “C₁-C₆-alkyl aryl” or “C₁-C₆-alkyl heteroaryl”.

“C₁-C₆-alkyl acyl” refers to C₁-C₆-alkyl groups having an acyl substituent, including 2-acetylethyl and the like.

“Aryl acyl” refers to aryl groups having an acyl substituent, including 2-acetylphenyl and the like.

“Heteroaryl acyl” refers to hetereoaryl groups having an acyl substituent, including 2-acetylpyridyl and the like.

“C₃-C₈-(hetero)cycloalkyl acyl” refers to 3 to 8 membered cycloalkyl or heterocycloalkyl groups having an acyl substituent.

“Acyloxy” refers to the group —OC(O)R where R includes H, “C₁-C₆-alkyl”, “C₂-C₆-alkenyl”, “C₂-C₆-alkynyl”, “C₃-C₈-cycloalkyl”, heterocycloalkyl “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acyloxy” refers to C₁-C₆-alkyl groups having an acyloxy substituent, including 2-(acetyloxy)ethyl and the like.

“Alkoxy” refers to the group —O—R where R includes “C₁-C₆-alkyl” or “aryl” or “heteroaryl” or “C₁-C₆-alkyl aryl” or “C₁-C₆-alkyl heteroaryl”. Preferred alkoxy groups include by way of example, methoxy, ethoxy, phenoxy and the like.

“C₁-C₆-alkyl alkoxy” refers to C₁-C₆-alkyl groups having an alkoxy substituent, including 2-ethoxyethyl and the like.

“Alkoxycarbonyl” refers to the group —C(O)OR where R includes H, “C₁-C₆-alkyl” or “aryl” or “heteroaryl” or “C₁-C₆-alkyl aryl” or “C₁-C₆-alkyl heteroaryl”.

“C₁-C₆-alkyl alkoxycarbonyl” refers to C₁-C₅-alkyl groups having an alkoxycarbonyl substituent, including 2-(benzyloxycarbonyl)ethyl and the like.

“Aminocarbonyl” refers to the group —C(O)NRR′ where each R, R′ includes independently hydrogen or C₁-C₆-alkyl or aryl or heteroaryl or “C₁-C₆-alkyl aryl” or “C₁-C₆-alkyl heteroaryl”.

“C₁-C₆-alkyl aminocarbonyl” refers to C₁-C₆-alkyl groups having an aminocarbonyl substituent, including 2-(dimethylaminocarbonyl)ethyl and the like.

“Acylamino” refers to the group —NRC(O)R′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl acylamino” refers to C₁-C₆-alkyl groups having an acylamino substituent, including 2-(propionylamino)ethyl and the like.

“Ureido” refers to the group —NRC(O)NR′R″ where each R, R′, R″ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”, and where R′ and R″, together with the nitrogen atom to which they are attached, can optionally form a 3-8-membered heterocycloalkyl ring.

“C₁-C₆-alkyl ureido” refers to C₁-C₆-alkyl groups having an ureido substituent, including 2-(N′-methylureido)ethyl and the like.

“Carbamate” refers to the group —NRC(O)OR′ where each R, R′ is independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“Amino” refers to the group —NRR′ where each R, R′ is independently hydrogen or “C₁-C₆-alkyl” or “aryl” or “heteroaryl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“C₁-C₆-alkyl amino” refers to C₁-C₅-alkyl groups having an amino substituent, including 2-(1-pyrrolidinyl)ethyl and the like.

“Ammonium” refers to a positively charged group —N⁺RR′R″, where each R, R′, R″ is independently “C₁-C₆-alkyl” or “C₁-C₆-alkyl aryl” or “C₁-C₆-alkyl heteroaryl”, or “cycloalkyl”, or “heterocycloalkyl”, and where R and R′, together with the nitrogen atom to which they are attached, can optionally form a 3-8-membered heterocycloalkyl ring.

“C₁-C₆-alkyl ammonium” refers to C₁-C₆-alkyl groups having an ammonium substituent, including 2-(1-pyrrolidinyl)ethyl and the like.

“Halogen” refers to fluoro, chloro, bromo and iodo atoms.

“Sulfonyloxy” refers to a group —OSO₂—R wherein R is selected from H, “C₁-C₆-alkyl”, “C₁-C₆-alkyl” substituted with halogens, e.g., an —OSO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyloxy” refers to C₁-C₅-alkyl groups having a sulfonyloxy substituent, including 2-(methylsulfonyloxy)ethyl and the like.

“Sulfonyl” refers to group “—SO₂R” wherein R is selected from H, “aryl”, “heteroaryl”, “C₁-C₆-alkyl”, “C₁-C₆-alkyl” substituted with halogens, e.g., an —SO₂—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonyl” refers to C₁-C₅-alkyl groups having a sulfonyl substituent, including 2-methylsulfonyl)ethyl and the like.

“Sulfinyl” refers to a group “—S(O)R” wherein R is selected from H, “C₁-C₆-alkyl”, “C₁-C₆-alkyl” substituted with halogens, e.g., a —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfinyl” refers to C₁-C₅-alkyl groups having a sulfinyl substituent, including 2-methylsulfinyl)ethyl and the like.

“Sulfanyl” refers to groups —S—R where R includes H, “C₁-C₆-alkyl”, “C₁-C₆-alkyl” substituted with halogens, e.g., a —SO—CF₃ group, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”. Preferred sulfanyl groups include methylsulfanyl, ethylsulfanyl, and the like.

“C₁-C₆-alkyl sulfanyl” refers to C₁-C₅-alkyl groups having a sulfanyl substituent, including 2-ethylsulfanyl)ethyl and the like.

“Sulfonylamino” refers to a group —NRSO₂—R′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl sulfonylamino” refers to C₁-C₅-alkyl groups having a sulfonylamino substituent, including 2-(ethylsulfonylamino)ethyl and the like.

“Aminosulfonyl” refers to a group —SO₂—NRR′ where each R, R′ includes independently hydrogen, “C₁-C₆-alkyl”, “C₂-C₆-alkenyl”, “C₂-C₆-alkynyl”, “C₃-C₈-cycloalkyl”, “heterocycloalkyl”, “aryl”, “heteroaryl”, “C₁-C₆-alkyl aryl” or “C₁-C₆-alkyl heteroaryl”, “C₂-C₆-alkenyl aryl”, “C₂-C₆-alkenyl heteroaryl”, “C₂-C₆-alkynyl aryl”, “C₂-C₆-alkynylheteroaryl”, “C₁-C₆-alkyl cycloalkyl”, “C₁-C₆-alkyl heterocycloalkyl”.

“C₁-C₆-alkyl aminosulfonyl” refers to C₁-C₆-alkyl groups having an aminosulfonyl substituent, including 2-(cyclohexylaminosulfonyl)ethyl and the like.

“Substituted or unsubstituted”: Unless otherwise constrained by the definition of the individual substituent, the above set out groups, like “alkyl”, “alkenyl”, “alkynyl”, “aryl” and “heteroaryl” etc. groups can optionally be substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “C₁-C₆-alkyl heteroaryl”, “C₁-C₆-alkyl cycloalkyl”, “C₁-C₆-alkyl heterocycloalkyl”, “amino”, “ammonium”, “acyl”, “acyloxy”, “acylamino”, “aminocarbonyl”, “alkoxycarbonyl”, “ureido”, “aryl”, “carbamate”, “heteroaryl”, “sulfinyl”, “sulfonyl”, “alkoxy”, “sulfanyl”, “halogen”, “carboxy”, trihalomethyl, cyano, hydroxy, mercapto, nitro, and the like. Alternatively said substitution could also comprise situations where neighbouring substituents have undergone ring closure, notably when vicinal functional substituents are involved, thus forming, e.g., lactams, lactams, cyclic anhydrides, but also acetals, thioacetals, animals formed by ring closure for instance in an effort to obtain a protective group.

“Pharmaceutically acceptable cationic salts or complexes” is intended to define such salts as the alkali metal salts, (e.g. sodium and potassium), alkaline earth metal salts (e.g. calcium or magnesium), aluminium salts, ammonium salts and salts with organic amines such as with methylamine, dimethyl amine, trimethyl amine, ethylamine, triethylamine, morpholine, N-Me-D-glucamine, N,N′-bis(phenylmethyl)-1,2-ethanediamine, ethanolamine, diethanolamine, ethylenediamine, N-methylmorpholine, piperidine, benzathine (N,N′-dibenzylethylenediamine), choline, ethylene-diamine, meglumine (N-methylglucamine), benethamine (N-benzylphenethylamine), diethylamine, piperazine, thromethamine (2-amino-2-hydroxymethyl-1,3-propanediol), procaine as well as amines of formula —NR, R′, R″ wherein R, R′, R″ is independently hydrogen, alkyl or benzyl. Especially preferred salts are sodium and potassium salts.

“Pharmaceutically acceptable salts or complexes” refers to salts or complexes of the below-identified compounds of the present invention that retain the desired biological activity. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. Said compounds can also be administered as pharmaceutically acceptable quaternary salts known by a person skilled in the art, which specifically include the quaternary ammonium salt of the formula —NR, R′, R″⁺ Z⁻, wherein R, R′, R″ is independently hydrogen, alkyl, or benzyl, C₁-C₆-alkyl, C₂-C₆-alkenyl, C₂-C₆-alkynyl, C₁-C₆-alkyl aryl, C₁-C₆-alkyl heteroaryl, cycloalkyl, heterocycloalkyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

“Pharmaceutically active derivative” refers to any compound that upon administration to the recipient, is capable of providing directly or indirectly, the activity disclosed herein.

“Enantiomeric excess” (ee) refers to the products that are obtained by an asymmetric synthesis, i.e. a synthesis involving non-racemic starting materials and/or reagents or a synthesis comprising at least one enantioselective step, whereby a surplus of one enantiomer in the order of at least about 52% ee is yielded.

General formula (I) according to the present invention also comprises its tautomers, its geometrical isomers, its optically active forms as enantiomers, diastereomers and its racemate forms, as well as pharmaceutically acceptable salts thereof. Preferred pharmaceutically acceptable salts of the formulae of the present invention are acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

The compounds of the present invention may be obtained as E/Z isomer mixture or as essentially pure E-isomers or Z isomers. The E/Z isomerism preferably refers to the vinyl moiety linking the phenyl with the azolidinone moiety. In a specific embodiment, the compounds of formula (I) are Z-isomers.

A first aspect of the present invention consists in the use of selective PI3 Kinase gamma inhibitor in the manufacture of a medicament for the treatment of disorders related to erythrocyte deficiency. Such PI3 Kinase gamma inhibitor compounds may be of formula (I)

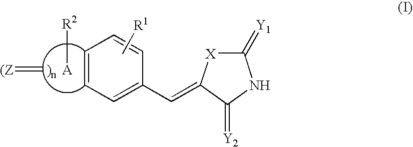

as well as its geometrical isomers, its optically active forms as enantiomers, diastereomers and its racemate forms, as well as pharmaceutically acceptable salts and pharmaceutically active derivatives thereof and may be used for the preparation of a medicament for the prophylaxis and/or treatment of disorders related to erythrocyte deficiency.

In a preferred embodiment, the selective PI3 Kinase gamma inhibitors are useful for the treatment and/or prophylaxis of haematological disorders including haemolytic anaemia, aplastic anaemia, pure red cell anaemia.

In a specific embodiment, the treatment of the haematological disorder comprises an initial or a simultaneous sensibilisation step using low amounts of erythropoetin (EPO) or a variant or analog thereof.

A further aspect of the present invention consists in a pharmaceutical composition comprising a PI3 Kinase gamma inhibitor and a pharmaceutically acceptable excipient. In a specific embodiment the pharmaceutical composition furthermore contains an erythropoetin (EPO), a variant or an analog thereof.

The pharmaceutical compositions of the present invention can be administered by a variety of routes including oral, rectal, transdermal, subcutaneous, intravenous, intramuscular and intranasal.

The administered dosage of EPO when combined with the simultaneous, preceding or subsequent administration of a PI3 Kinase gamma inhibitor usually does not exceed about 300 IU/kg of body weight, more preferably 250 IU/kg of body weight, even more preferably not more than 250, 150, 75 or 50 IU/kg of body weight.

The substituents within formula (I) are defined as follows:

A is an unsubstituted or substituted 5-8 membered heterocyclic group or an unsubstituted or substituted carbocyclic group.

Said carbocyclic group may be fused with an unsubstituted or substituted aryl, an unsubstituted or substituted heteroaryl, an unsubstituted or substituted cycloalkyl or an unsubstituted or substituted heterocycloalkyl.

Such heterocyclic or carbocyclic groups comprise aryl, heteroaryl, cycloalkyl and heterocycloalkyl, including phenyl, phenantrenyl, cyclopentyl, cyclohexyl, norbornyl, pyrrolidine, piperidine, piperazine, 1-methylpiperazine, morpholine, pyrrolyl, furanyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl or benzoquinolyl

Further examplary heterocyclic or carbocyclic groups A include unsubstituted or substituted dioxol, unsubstituted or substituted dioxin, unsubstituted or substituted dihydrofuran, unsubstituted or substituted (dihydro) furanyl, unsubstituted or substituted (dihydro)oxazinyl, unsubstituted or substituted oxazinoyl, unsubstituted or substituted pyridinyl, unsubstituted or substituted isooxazolyl, unsubstituted or substituted oxazolyl unsubstituted or substituted (dihydro)napthalenyl, unsubstituted or substituted pyrimidinyl, unsubstituted or substituted triazolyl, unsubstituted or substituted imidazolyl, unsubstituted or substituted pyrazinyl, unsubstituted or substituted thiazolyl, unsubstituted or substituted thiadiazolyl, unsubstituted or substituted oxadiazolyl.

X is S, O or NH, preferably S.

Y¹ and Y² are independently from each other selected from the group consisting of S, O or —NH, preferably O.

Z is S or O, preferably O.

R¹ is selected from the group comprising or consisting of H, CN, carboxy, acyl, C₁-C₆-alkoxy, halogen, hydroxy, acyloxy, an unsubstituted or substituted C₁-C₆-alkyl carboxy, an unsubstituted or substituted C₁-C₆-alkyl acyloxy, an unsubstituted or substituted C₁-C₆-alkyl alkoxy, alkoxycarbonyl, an unsubstituted or substituted C₁-C₆-alkyl alkoxycarbonyl, aminocarbonyl, an unsubstituted or substituted C₁-C₆-alkyl aminocarbonyl, acylamino, an unsubstituted or substituted C₁-C₆-alkyl acylamino, ureido, an unsubstituted or substituted C₁-C₆-alkyl ureido, amino, an unsubstituted or substituted C₁-C₆-alkyl amino, ammonium, sulfonyloxy, an unsubstituted or substituted C₁-C₆-alkyl sulfonyloxy, sulfonyl, an unsubstituted or substituted C₁-C₆-alkyl sulfonyl, sulfinyl, an unsubstituted or substituted C₁-C₆-alkyl sulfinyl, sulfanyl, an unsubstituted or substituted C₁-C₆-alkyl sulfanyl, sulfonylamino, an unsubstituted or substituted C₁-C₆-alkyl sulfonylamino or carbamate. In a specific embodiment R¹ is H.

R² is selected from the group comprising or consisting of H, halogen, acyl, amino, an unsubstituted or substituted C₁-C₆-alkyl, an unsubstituted or substituted C₂-C₆-alkenyl, an unsubstituted or substituted C₂-C₆-alkynyl, an unsubstituted or substituted C₁-C₆-alkyl carboxy, an unsubstituted or substituted C₁-C₆-alkyl acyl, an unsubstituted or substituted C₁-C₆-alkyl alkoxycarbonyl, an unsubstituted or substituted C₁-C₆-alkyl aminocarbonyl, an unsubstituted or substituted C₁-C₆-alkyl acyloxy, an unsubstituted or substituted C₁-C₆-alkyl acylamino, an unsubstituted or substituted C₁-C₆-alkyl ureido, an unsubstituted or substituted C₁-C₆-alkyl carbamate, an unsubstituted or substituted C₁-C₆-alkyl amino, an unsubstituted or substituted C₁-C₆-alkyl alkoxy, an unsubstituted or substituted C₁-C₆-alkyl sulfanyl, an unsubstituted or substituted C₁-C₆-alkyl sulfinyl, an unsubstituted or substituted C₁-C₆-alkyl sulfonyl, an unsubstituted or substituted C₁-C₆-alkyl sulfonylaminoaryl, an unsubstituted or substituted aryl, an unsubstituted or substituted C₃-C₈-cycloalkyl or heterocycloalkyl, an unsubstituted or substituted C₁-C₆-alkyl aryl, an unsubstituted or substituted C₂-C₆-alkenyl-aryl, an unsubstituted or substituted C₂-C₆-alkynyl aryl, carboxy, cyano, hydroxy, C₁-C₆-alkoxy, nitro, acylamino, ureido, sulfonylamino, sulfanyl, or sulfonyl.

n is an integer 0, 1 or 2, preferably n is 0 or 1. Most preferred is n=0.

According to a specific embodiment of the invention, R¹ and R² are both H.

In a further specific embodiment according to the invention, X is S, Y¹ and Y² are both O, R¹ and R² are as above defined and n is 0.

A specific sub-group of formula (I) are compounds having the formula (Ic), whereby R¹, Y¹ are as above defined and W is O or S; specifically R¹ may be an unsubstituted or substituted C₁-C₄ alkyl group or an unsubstituted or substituted C₁-C₅ alkenyl group, carboxy, cyano, C₁-C₄-alkoxy, nitro, acylamino, ureido.

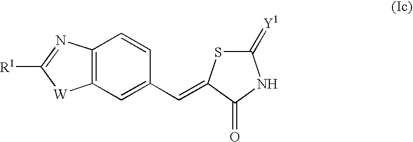

Still further compounds have the formula (II-a)

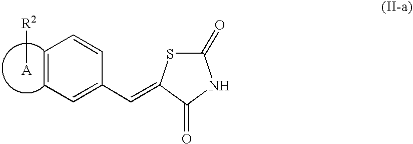

A is selected from the group consisting of unsubstituted or substituted dioxol, unsubstituted or substituted dioxin, unsubstituted or substituted dihydrofuran, unsubstituted or substituted (dihydro) furanyl, unsubstituted or substituted (dihydro)oxazinyl, unsubstituted or substituted oxazinoyl, unsubstituted or substituted pyridinyl, unsubstituted or substituted isooxazolyl, unsubstituted or substituted oxazolyl unsubstituted or substituted (dihydro)napthalenyl, unsubstituted or substituted pyrimidinyl, unsubstituted or substituted triazolyl, unsubstituted or substituted imidazolyl, unsubstituted or substituted pyrazinyl, unsubstituted or substituted thiazolyl, unsubstituted or substituted thiadiazolyl, unsubstituted or substituted oxadiazolyl.

R² is selected from the group comprising or consisting of H, halogen, acyl, amino, unsubstituted or substituted C₁-C₆-alkyl, unsubstituted or substituted C₂-C₆-alkenyl, unsubstituted or substituted C₂-C₆-alkynyl, unsubstituted or substituted C₁-C₆-alkyl carboxy, unsubstituted or substituted C₁-C₆-alkyl acyl, unsubstituted or substituted C₁-C₆-alkyl alkoxycarbonyl, unsubstituted or substituted C₁-C₆-alkyl aminocarbonyl, unsubstituted or substituted C₁-C₆-alkyl acyloxy, unsubstituted or substituted C₁-C₆-alkyl acylamino, unsubstituted or substituted C₁-C₆-alkyl ureido, unsubstituted or substituted C₁-C₆-alkyl carbamate, unsubstituted or substituted C₁-C₆-alkyl amino, unsubstituted or substituted C₁-C₆-alkyl alkoxy, unsubstituted or substituted C₁-C₆-alkyl sulfanyl, unsubstituted or substituted C₁-C₆-alkyl sulfinyl, unsubstituted or substituted C₁-C₆-alkyl sulfonyl, unsubstituted or substituted C₁-C₆-alkyl sulfonylaminoaryl, an unsubstituted or substituted aryl, unsubstituted or substituted C₃-C₈-cycloalkyl or heterocycloalkyl, unsubstituted or substituted C₁-C₆-alkyl aryl, unsubstituted or substituted C₂-C₆-alkenylaryl, unsubstituted or substituted C₂-C₆-alkynyl aryl, carboxy, cyano, hydroxy, C₁-C₆-alkoxy, nitro, acylamino, ureido, sulfonylamino, sulfanyl, or sulfonyl.

More specific thiazolidinone-vinyl fused-benzene derivatives are of formula (II)

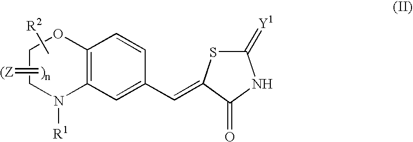

as well as its geometrical isomers, its optically active forms as enantiomers, diastereomers and its racemate forms, as well as pharmaceutically acceptable salts and pharmaceutically active derivatives thereof, wherein Y¹, Z, R¹, R² are as above defined and n is 0 or 1.

In a specific embodiment R¹ is an unsubstituted or substituted C₁-C₆-alkyl, an unsubstituted or substituted C₁-C₆-alkyl aryl, an unsubstituted or substituted aryl, an unsubstituted or substituted C₃-C₈-cycloalkyl or -heterocycloalkyl, an unsubstituted or substituted C₁-C₆-alkyl aryl, an unsubstituted or substituted C₂-C₆-alkenyl-aryl, an unsubstituted or substituted C₂-C₆-alkynyl aryl.

In a specific embodiment Y¹ is O,

Still further thiazolidinone-vinyl fused-benzene derivatives are of formula (III)

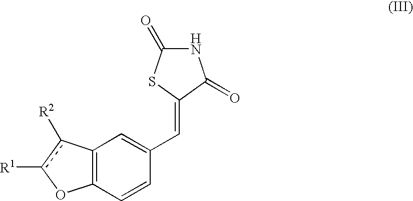

as well as its geometrical isomers, its optically active forms as enantiomers, diastereomers and its racemate forms, as well as pharmaceutically acceptable salts and pharmaceutically active derivatives thereof, wherein R¹ and R² are as above defined (the dotted line represents the optional presence of a double bond).

Still a further embodiment comprises compounds of formulae (IV), (V) and (VI):

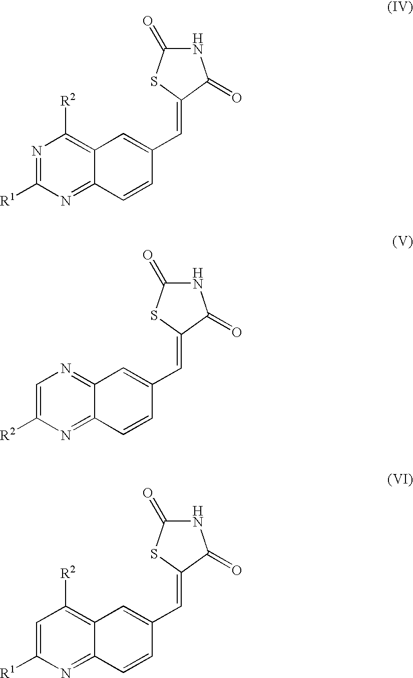

R¹ is selected from the group consisting of hydrogen, halogen, cyano, C₁-C₆-alkyl, C₁-C₆-alkoxy, acyl, alkoxy carbonyl, while R² is as above defined. In a specific embodiment R² is an amino moiety.

Still a further embodiment comprises compounds of formula (I′)

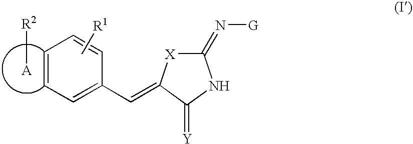

A is an unsubstituted or substituted 5-8 membered heterocyclic group or an unsubstituted or substituted carbocyclic group. Preferably, A is a heterocyclic moiety.

In one embodiment of the present invention A is a dioxolenyl or a pyridinyl moiety.

X is S, O or —NR³, preferably S. R³ is selected from the group comprising or consisting of H or C₁-C₆-alkyl.

Y is S or O, preferably O.

R¹ is selected from the group comprising or consisting of H, CN, carboxy, acyl, C₁-C₆-alkoxy, halogen, hydroxy, acyloxy, an unsubstituted or substituted C₁-C₆-alkyl carboxy, an unsubstituted or substituted C₁-C₆-alkyl acyloxy, an unsubstituted or substituted C₁-C₆-alkyl alkoxy, alkoxycarbonyl, an unsubstituted or substituted C₁-C₆-alkyl alkoxycarbonyl, aminocarbonyl, an unsubstituted or substituted C₁-C₆-alkyl aminocarbonyl, acylamino, an unsubstituted or substituted C₁-C₆-alkyl acylamino, ureido, an unsubstituted or substituted C₁-C₆-alkyl ureido, amino, an unsubstituted or substituted C₁-C₆-alkyl amino, ammonium, sulfonyloxy, an unsubstituted or substituted C₁-C₆-alkyl sulfonyloxy, sulfonyl, an unsubstituted or substituted C₁-C₆-alkyl sulfonyl, sulfinyl, an unsubstituted or substituted C₁-C₆-alkyl sulfinyl, sulfanyl, an unsubstituted or substituted C₁-C₆-alkyl sulfanyl, sulfonylamino, an unsubstituted or substituted C₁-C₆-alkyl sulfonylamino or carbamate. Preferably R¹ is H.

R² is selected from the group comprising or consisting of H, halogen, acyl, amino, an unsubstituted or substituted C₁-C₆-alkyl, an unsubstituted or substituted C₂-C₆-alkenyl, an unsubstituted or substituted C₂-C₆-alkynyl, an unsubstituted or substituted C₁-C₆-alkyl carboxy, an unsubstituted or substituted C₁-C₆-alkyl acyl, an unsubstituted or substituted C₁-C₆-alkyl alkoxycarbonyl, an unsubstituted or substituted C₁-C₆-alkyl aminocarbonyl, an unsubstituted or substituted C₁-C₆-alkyl acyloxy, an unsubstituted or substituted C₁-C₆-alkyl acylamino, an unsubstituted or substituted C₁-C₆-alkyl ureido, an unsubstituted or substituted C₁-C₆-alkyl carbamate, an unsubstituted or substituted C₁-C₆-alkyl amino, an unsubstituted or substituted C₁-C₆-alkyl alkoxy, an unsubstituted or substituted C₁-C₆-alkyl sulfanyl, an unsubstituted or substituted C₁-C₆-alkyl sulfinyl, an unsubstituted or substituted C₁-C₆-alkyl sulfonyl, an unsubstituted or substituted C₁-C₆-alkyl sulfonylaminoaryl, aryl, heteroaryl, an unsubstituted or substituted C₃-C₈-cycloalkyl or heterocycloalkyl, an unsubstituted or substituted C₁-C₆-alkyl aryl, an unsubstituted or substituted C₁-C₆-alkyl heteroaryl, an unsubstituted or substituted C₂-C₆-alkenyl-aryl or -heteroaryl, an unsubstituted or substituted C₂-C₆-alkynyl aryl or -heteroaryl, carboxy, cyano, hydroxy, C₁-C₆-alkoxy, nitro, acylamino, ureido, sulfonylamino, sulfanyl, or sulfonyl. Preferably R² is H.

In a specific embodiment, R¹ and R² are both H.

G is a substituted or unsubstituted C₁-C₆-alkyl, substituted or unsubstituted C₂-C₆-alkyenyl, substituted or unsubstituted C₂-C₆-alkynyl, substituted or unsubstituted heteroaryl, an unsubstituted or substituted C₁-C₆-alkyl aryl, an unsubstituted or substituted C₁-C₆-alkyl heteroaryl, an unsubstituted or substituted C₂-C₆-alkenyl-aryl or -heteroaryl, an unsubstituted or substituted C₂-C₆-alkynyl aryl or -heteroaryl, substituted or unsubstituted C₁-C₆-alkoxy, cyano, substituted or unsubstituted C₁-C₆-acyl or G is a sulfonyl moiety.

In particular, G is selected from the group comprising or consisting of a sulfonyl moiety, a cyano or an substituted or unsubstituted C₁-C₆-alkoxy.

Specific compounds are:

### Example Name

1 5-(1,3-benzodioxol-5-ylmethylene)-1,3-thiazolidine-2,4-dione
2 5-(1,3-benzodioxol-5-ylmethylene)-2-thioxo-1,3-thiazolidin-4-one
3 5-(2,3-dihydro-1,4-benzodioxin-6-ylmethylene)-1,3-thiazolidine-2,4-dione
4 5-[(9,10-dioxo-9,10-dihydroanthracen-2-yl)methylene]-1,3-thiazolidine-2,4-dione
5 (5-[(2,2-difluoro-1,3-benzodioxol-5-yl)methylene]-1,3-thiazolidine-2,4-dione
6 5-[(4-methyl-3-oxo-3,4-dihydro-2H-1,4-benzoxazin-6-yl)methylene]-1,3-thiazolidine-2,4-dione
7 5-(1,3-benzodioxol-5-ylmethylene)-2-imino-1,3-thiazolidin-4-one
8 5-(3-Methyl-benzofuran-5-ylmethylene)-thiazolidine-2,4-dione
9 3-[5-(2,4-Dioxo-thiazolidin-5-ylidenemethyl)benzofuran-3-yl]-acrylic acid
10 5-(3,4-Dihydro-2H-benzo[1,4]oxazin-6-ylmethylene)-thiazolidine-2,4-dione
11 5-(3-Amino-benzo[d]isoxazol-5-ylmethylene)thiazolidine-2,4-dione
12 5-Benzo[1,2,5]oxadiazol-5-ylmethylene-thiazolidine-2,4-ione
13 5-(2-Fluoro-benzofuran-6-ylmethylene)-thiazolidine-2,4-dione
14 5-Benzo[1,3]dioxol-5-ylmethylene-4-oxo-thiazolidin-2-ylidene-cyanamide

The compounds of formula (I) may be obtained according to the methods described in PCT/EP02/100798 and EP-03102313.8

The pharmaceutical compositions of the present invention typically comprise a pharmaceutically acceptable carrier, diluent or excipient. A person skilled in the art is aware of a whole variety of such carrier, diluent or excipient compounds suitable to formulate a pharmaceutical composition.

The medicament of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

The pharmaceutical compositions of the present invention can be administered by a variety of routes including oral, rectal, transdermal, subcutaneous, intravenous, intramuscular and intranasal. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the PI3 Kinase gamma inhibitor is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like.

Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As above mentioned, the PI3 Kinase gamma inhibitor in such compositions is typically a minor component, frequently ranging between 0.05 to 10% by weight with the remainder being the injectable carrier and the like. The injectable may also contain EPO or a variant or an analog

The above described components for orally administered or injectable compositions are merely representative. Further materials as well as processing techniques and the like are set out in Part 5 of Remington's Pharmaceutical Sciences, 20^{th} Edition, 2000, Marck Publishing Company, Easton, Pa., which is incorporated herein by reference.

The compounds of this invention can also be administered in sustained release forms or from sustained release drug delivery systems. A description of representative sustained release materials can also be found in the incorporated materials in Remington's Pharmaceutical Sciences.

### EXAMPLE 15

### Biological Assays

The compounds used according to the present invention are selective inhibitors of PI3 Kinase gamma. Thus, the compounds are at least twice as active in inhibiting PB Kinase gamma than in inhibiting PI3 Kinase alpha or delta. More preferably they are 4 times, even more preferably more than 6 times more active in inhibiting PI3 Kinase gamma than in inhibiting PI3 Kinase alpha or delta.

To assess the compounds in terms of their selectivity with respect to the isoforms of PI3 Kinase, they may be subjected to the following binding assay:

### a) High Throughput PI3K Lipid Kinase Assay (Binding Assay):

The assay combines the scintillation proximity assay technology (SPA, Amersham) with the capacity of neomycin (a polycationic antibiotic) to bind phospholipids with high affinity and specificity. The Scintillation Proximity Assay is based on the properties of weakly emitting isotopes (such as ³H, ¹²⁵I, ³³P). Coating SPA beads with neomycin allows the detection of phosphorylated lipid substrates after incubation with recombinant PI3K and radioactive ATP in the same well, by capturing the radioactive phospholipids to the SPA beads through their specific binding to neomycin.

To a 384 wells MTP containing 5 μl of the test compound of formula (I) (solubilized in 6% DMSO; to yield a concentration of 100, 30, 10, 3, 1, 0.3, 0.1, 0.03, 0.01, 0.001 μM of the test compound), the following assay components are added. 1) 5 μl (58 ng) of Human recombinant GST-PI3Kγ or GST-PI3Kα or GST-PI3Kδ (in Hepes 40 mM, pH 7.4, DTT 1 mM and ethylenglycol 5%) 2) 10 μl of lipid micelles and 3) 10 μl of Kinase buffer ([³³P]γ-ATP 45 μM/60 nCi, MgCl₂ 30 mM, DTT 1 mM, β-Glycerophosphate 1 mM, Na₃VO₄ 100 μM, Na Cholate 0.3%, in Hepes 40 mM, pH 7.4). After incubation at room temperature for 180 minutes, with gentle agitation, the reaction is stopped by addition of 60 μl of a solution containing 100 μg of neomycin-coated PVT SPA beads in PBS containing ATP 10 mM and EDTA 5 mM. The assay is further incubated at room temperature for 60 minutes with gentle agitation to allow binding of phospholipids to neomycin-SPA beads. After precipitation of the neomycin-coated PVT SPA beads for 5 minutes at 1500×g, radioactive PtdIns(3)P is quantified by scintillation counting in a Wallac MicroBeta™ plate counter.

The values indicated in respect of PI3K γ or PI3K δ or GST-PI3K α refer to the IC₅₀ (μM), i.e. the amount necessary to achieve 50% inhibition of said target.

Example compounds are set out in Table 1.

TABLE 1
IC₅₀ values of selective PI3Kγ Inhibitors

| Example | PI3Kγ, IC₅₀(μM) | PI3Kα, IC₅₀(μM) | PI3Kδ, IC₅₀(μM) |
|---|---|---|---|
| 1 | 0.070 | 0.25 | 1.70 |
| 2 | 0.115 | 0.45 | 20 |
| 3 | 0.050 | 0.31 | 20 |
| 4 | 0.316 | 1.37 | 4.87 |
| 5 | 0.250 | 12 | 20 |
| 6 | 0.03 | 0.17 | 20 |
| 7 | 0.71 | 20 | 20 |
| 8 | 0.03 | 0.135 | 1.97 |
| 9 | 0.008 | 0.032 | 2.48 |
| 10 | 0.59 | 2.1 | 20 |
| 11 | 0.095 | 0.46 | 10 |
| 12 | 0.035 | 0.37 | 2.73 |
| 13 | 0.29 | 1.2 | 20 |
| 14 | 0.021 | 0.231 | 1.17 |

### b) EPO Induced Erythrocyte Formation:

Investigation of the effect of PI3K inhibitors on the differentiation of red cell population, but also on the expansion of undifferentiated stem cells. Two different culture systems. A and B, were explored.

### Culture System A (Containing No IL-3):

Differentiation of bone-marrow derived stem cells into the red cell line only. Semi-optimal systems in respect of the expansion of colony forming units, because of the lack of IL3 and other early acting growth factors.

The culture conditions are: IMDM-culture medium, 30% pre-selected fetal calf serum, 1% BSA, Glutamine 40 ug/ml iron saturated transferin, 10−6 Mol Mercaptoethanol, 10 ng/ml SCF, 100 U/ml IL6, 7 U Epo.

### Culture System B (Containing IL-3):

Colony forming unit assay. The culture conditions are: IMDM-culture medium, 30% pre-selected fetal calf serum, 1% BSA, Glutamine, 40 ug/ml iron saturated transferin, 10−6 Mol Mercaptoethanol, 10 ng/ml SCF, 100 U/ml IL6, 7 U/ml Epo, 100 U/ml IL3, 28 ng/ml GM-CSF, 0.3% agar.

Mobilized CD34 positive stem cells were seeded into the culture at day 0 and expanded for 13 days. Samples for the colony assay or for flow cytometry were taken at day 0, 3, 6 9 and 13.

The colony assay was first performed in the absence of the test compounds of formula (I) in order to determine the CFU rate (colony forming unit). After 13 days the number of colonies was assesses. In a second run the same assay was performed using a test compound according to formula (I) added to the culture systems at day 1.

PI-3Kγ is up-regulated during the first 12 h of in vitro culture of CD34 positive stem cells and down regulated following 5 days of Epo treatment. Thereby, the effect of the test compounds according to formula (I) on the expansion of hematopoietic progenitor cells and on the red cell differentiation was investigated.

Colony forming units and the expression of Glycophorin A (marker) on the cell surface were used as markers for stem cell erythropoesis. Additionally, the presence of the cell surface antigen CD34 was also monitored to assess the differentiation status of the in vitro expanded cell lineage.

Treatment of cells with test compounds according to formula (I) resulted in a significantly higher expansion rate of nucleated cells compared to the untreated control. For instance the use of the compound of Example 1 (i.e. 5-(1,3-benzodioxol-5-ylmethylene)-1,3-thiazolidine-2,4-dione) results in a 4-fold increase of the expansion over the control in 13 days. At the same time, the cell proliferation was not enhanced.

Relative expansion rate of GlyA-positive cells: At day 0, the stage of differentiation only few, if any cells, are GlyA positive. This antigen is induced by the binding of the Epo-receptor and to a lesser extent by the TPO-receptor (thrombocyte). The expansion rate is calculated as the absolute number of GlyA-positive cells at the time point of interest divided by the absolute number of GlyA-positive cells in the starting material.

Pharmacological inhibition of PI-3Kγ using the test compounds according to formula (I) resulted in an accelerated up-regulation of GlyA surface marker and hence an increased proliferation of those cells.

The growth factor combination SCF, IL-6 and Epo is not sufficient for an expansion of early hematopoietic cell. This results in a low expansion of those cells. But even under those sub-optimal conditions the inhibition of PI-3Kγ caused a higher expansion of the CD34-positive cells

An increase of the GlyA-antigen expression on CD34-positive cells may suggest an influence of PI-3Kγ on the later differentiation steps but also at the progenitor level. This can be confirmed by the analysis of the erythroid colony forming units (CFU-E) and burst forming units (BFU).

The absolute expansion rate of CFU-GM:

Myeloid progenitor cells are not supported by the selected growth factor combination. Consequently, the CFU-GM expansion is very low compared to a medium complemented by SCF, IL-3, IL-6, GM-CSF and Epo.

### REFERENCES LIST

1. Vanhaesebroeck B et al., Trends Biochem Sci. 22(7) p. 267-72 (1997);
2. Leslie N. R et al Chem. Rev. 101(8) p. 2365-80 (2001);
3. Katso R. et al. Annu Rev Cell Dev Biol. 17 p. 615-75 (2001)
4. Toker a. et al Cell Mol Life Sci. 59(5) p. 761-79 (2002);
5. Vanhaesebroeck B. Exp Cell Res. 25(1) p. 239-54 (1999)
6. June H. Myklebust et al., Experimental Hematology 30 (2002), 990
7. L. Neri et al. Cellular Signalling 14 (2002) 21
8. PCT/EP02/100798
9. EP-03102313.8

1-18. (canceled)

19. A method of treating a subject with a disorder related to erythrocyte deficiency, comprising administering to the subject an effective amount of a selective PI3 Kinase gamma inhibitor.

20. The method according to claim 19, further comprising administering to the subject an effective amount of erythropoetin (EPO), a variant, or an analogue thereof.

21. The method according claim 19, wherein the disease is anaemia.

22. The method according to claim 21, wherein the disease is haemolytic anaemia, aplastic anaemia, or pure red cell anaemia.

23. The method according to claim 19, wherein the PI3 Kinase gamma inhibitor is a compound according to formula (I) 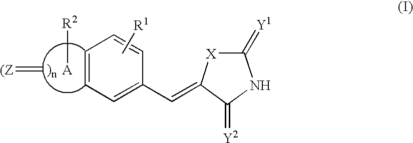
as well as its geometrical isomers, its optically active forms as enantiomers, diastereomers and its racemate forms, as well as pharmaceutically acceptable salts and pharmaceutically active derivatives thereof, wherein
A is a 5-8 membered heterocyclic or carbocyclic group, wherein said carbocyclic group may be fused with aryl, heteroaryl, cycloalkyl or heterocycloalkyl;
X is S, O or NH;
Y¹ and Y² are independently S, O or —NH;
Z is S or O;
R¹ is H, CN, carboxy, acyl, C₁-C₆ alkoxy, halogen, hydroxy, acyloxy, C₁-C₆-alkyl carboxy, C₁-C₆-alkyl acyloxy, C₁-C₆-alkyl alkoxy, alkoxycarbonyl, C₁-C₆-alkyl alkoxycarbonyl, aminocarbonyl, C₁-C₆-alkyl aminocarbonyl, acylamino, C₁-C₆-alkyl acylamino, ureido, C₁-C₆-alkyl ureido, amino, C₁-C₆-alkyl amino, ammonium, sulfonyloxy, C₁-C₆-alkyl sulfonyloxy, sulfonyl, C₁-C₆-alkyl sulfonyl, sulfinyl, C₁-C₆-alkyl sulfinyl, sulfanyl, C₁-C₆-alkyl sulfanyl, sulfonylamino, C₁-C₆-alkyl sulfonylamino or carbamate;
R² is selected from the group comprising or consisting of H, halogen, acyl, amino, C₁-C₆-alkyl, C₂-C₆-alkenyl, C₂-C₆-alkynyl, C₁-C₆-alkyl carboxy, C₁-C₆-alkyl acyl, C₁-C₆-alkyl alkoxycarbonyl, C₁-C₆-alkyl aminocarbonyl, C₁-C₆-alkyl acyloxy, C₁-C₆-alkyl acylamino, C₁-C₆-alkyl ureido, C₁-C₆-alkyl amino, C₁-C₆-alkyl alkoxy, C₁-C₆-alkyl sulfanyl, C₁-C₆-alkyl sulfinyl, C₁-C₆-alkyl sulfonyl, C₁-C₆-alkyl sulfonylaminoaryl, aryl, C₃-C₈-cycloalkyl or heterocycloalkyl, C₁-C₆-alkyl aryl, C₂-C₆-alkenyl-aryl, C₂-C₆-alkynyl aryl, carboxy, cyano, hydroxy, C₁-C₆-alkoxy, nitro, acylamino, ureido, C₁-C₆-alkyl carbamate, sulfonylamino, sulfanyl, or sulfonyl;
n is 0, 1 or 2.

24. The method according to claim 23, wherein Y¹ and Y² are both oxygen.

25. The method according to claim 23, wherein n is 1 or 2 and R¹ and R² are both H.

26. The method according to claim 23, wherein X is S, Y¹ and Y² are O, R¹ and R² are both H and n is 0.

27. The method according to claim 23, wherein the PI3 Kinase gamma inhibitor is a compound according to formula (II-a) 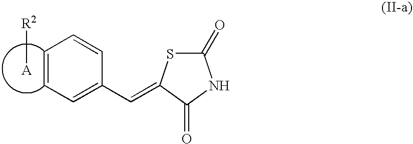
or a pharmaceutically acceptable salt thereof, wherein:
A is selected from the group consisting of dioxol, dioxin, dihydrofuran, (dihydro) furanyl, (dihydro)oxazinyl, pyridinyl, isooxazolyl, oxazolyl (dihydro)napthalenyl, pyrimidinyl, triazolyl, imidazolyl, pyrazinyl, thiazolidinyl, thiadiazolyl, oxadiazolyl;
R² is selected from the group comprising or consisting of H, halogen, acyl, amino, C₁-C₆-alkyl, C₂-C₆-alkenyl, C₂-C₆-alkynyl, C₁-C₆-alkyl carboxy, C₁-C₆-alkyl acyl, C₁-C₆-alkyl alkoxycarbonyl, C₁-C₆-alkyl aminocarbonyl, C₁-C₆-alkyl acyloxy, C₁-C₆-alkyl acylamino, C₁-C₆-alkyl ureido, C₁-C₆-alkyl carbamate, C₁-C₆-alkyl amino, C₁-C₆-alkyl alkoxy, C₁-C₆-alkyl sulfanyl, C₁-C₆-alkyl sulfinyl, C₁-C₆-alkyl sulfonyl, C₁-C₆-alkyl sulfonylaminoaryl, aryl, C₃-C₈-cycloalkyl or heterocycloalkyl, C₁-C₆-alkyl aryl, C₂-C₆-alkenyl-aryl, C₂-C₆-alkynyl aryl, carboxy, cyano, hydroxy, C₁-C₆-alkoxy, nitro, acylamino, ureido, sulfonylamino, sulfanyl, or sulfonyl.

28. The method according to claim 23, wherein the PI3 Kinase gamma inhibitor is a compound according to formula (II) 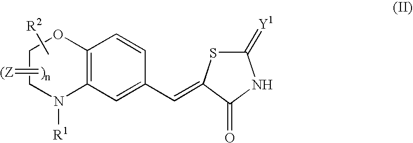
as well as its geometrical isomers, its optically active forms as enantiomers, diastereomers and its racemate forms, as well as pharmaceutically acceptable salts and pharmaceutically active derivatives thereof, wherein n is 0 or 1.

29. The method according to claim 28, wherein Y¹ is O.

30. The method according to claim 28, wherein R¹ is selected from the group consisting of C₁-C₆-alkyl, C₁-C₆-alkyl aryl, aryl, C₃-C₈-cycloalkyl or heterocycloalkyl, C₁-C₆-alkyl aryl, C₂-C₆-alkenyl-aryl and C₂-C₆-alkynyl aryl.

31. The method according to claim 23, wherein the PI3 Kinase gamma inhibitor is a compound according to formula (III) 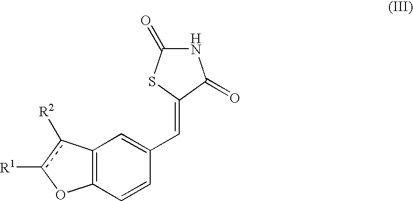
as well as its geometrical isomers, its optically active forms as enantiomers, diastereomers and its racemate forms, as well as pharmaceutically acceptable salts and pharmaceutically active derivatives thereof.

32. The method according to claim 23, wherein the PI3 Kinase gamma inhibitor is a compound according any of the formulae (IV), (V) and (VI) 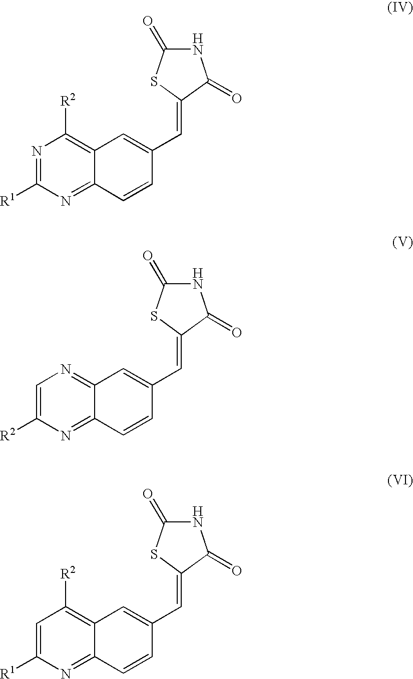
or a pharmaceutically acceptable salt thereof;
wherein R¹ is selected from the group consisting of hydrogen, halogen, cyano, C₁-C₆-alkyl, C₁-C₆-alkoxy, acyl, alkoxy carbonyl.

33. The method according to claim 19, wherein the PI3 Kinase gamma inhibitor is a compound to formula (I′), 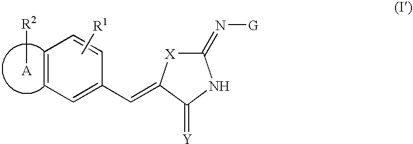
or a pharmaceutically acceptable salt thereof;
wherein A is an 5-8 membered heterocyclic group or an carbocyclic group which may be fused with an aryl, an heteroaryl, an cycloalkyl or an heterocycloalkyl;
X is S, O or —NR³;
Y is S or O;
R¹ is selected from the group comprising or consisting of H, CN, carboxy, acyl, C₁-C₆-alkoxy, halogen, hydroxy, acyloxy, C₁-C₆-alkyl carboxy, C₁-C₆-alkyl acyloxy, C₁-C₆-alkyl alkoxy, alkoxycarbonyl, C₁-C₆-alkyl alkoxycarbonyl, aminocarbonyl, C₁-C₆-alkyl aminocarbonyl, acylamino, C₁-C₆-alkyl acylamino, ureido, C₁-C₆-alkyl ureido, amino, C₁-C₆-alkyl amino, ammonium, sulfonyloxy, C₁-C₆-alkyl sulfonyloxy, sulfonyl, C₁-C₆-alkyl sulfonyl, sulfinyl, C₁-C₆-alkyl sulfinyl, sulfanyl, C₁-C₆-alkyl sulfanyl, sulfonylamino, C₁-C₆-alkyl sulfonylamino or carbamate;
R² is selected from the group comprising or consisting of H, halogen, acyl, amino, C₁-C₆-alkyl, C₂-C₆-alkenyl, C₂-C₆-alkynyl, C₁-C₆-alkyl carboxy, C₁-C₆-alkyl acyl, C₁-C₆-alkyl alkoxycarbonyl, C₁-C₆-alkyl aminocarbonyl, C₁-C₆-alkyl acyloxy, C₁-C₆-alkyl acylamino, C₁-C₆-alkyl ureido, C₁-C₆-alkyl carbamate, C₁-C₆-alkyl amino, C₁-C₆-alkyl alkoxy, C₁-C₆-alkyl sulfanyl, C₁-C₆-alkyl sulfinyl, C₁-C₆-alkyl sulfonyl, C₁-C₆-alkyl sulfonylaminoaryl, aryl, heteroaryl, C₃-C₈-cycloalkyl or heterocycloalkyl, C₁-C₆-alkyl aryl, C₁-C₆-alkyl heteroaryl, C₂-C₆-alkenyl-aryl or -heteroaryl, C₂-C₆-alkynyl aryl or -heteroaryl, carboxy, cyano, hydroxy, C₁-C₆-alkoxy, nitro, acylamino, ureido, sulfonylamino, sulfanyl, or sulfonyl;
G is a C₁-C₆-alkyl, C₂-C₆-alkyenyl, C₂-C₆-alkynyl, heteroaryl, C₁-C₆-alkyl aryl, C₁-C₆-alkyl heteroaryl, C₂-C₆-alkenyl-aryl or -heteroaryl, C₂-C₆-alkynyl aryl or -heteroaryl, C₁-C₆-alkoxy, cyano, C₁-C₆-acyl, or a sulfonyl moiety.
R³ is selected from the group comprising or consisting of H or C₁-C₆-alkyl.

34. The method according to claim 19, wherein the PI3 Kinase gamma inhibitor is a compound selected from the group consisting of:
5-(1,3-benzodioxol-5-ylmethylene)-1,3-thiazolidine-2,4-dione;
5-(1,3-benzodioxol-5-ylmethylene)-2-thioxo-1,3-thiazolidin-4-one;
5-(2,3-dihydro-1,4-benzodioxin-6-ylmethylene)-1,3-thiazolidine-2,4-dione;
5-(2,3-dihydro-1-benzofuran-5-ylmethylene)-1,3-thiazolidine-2,4-dione;
5-[(7-methoxy-1,3-benzodioxol-5-yl)methylene]-1,3-thiazolidine-2,4-dione;
5-[(9,10-dioxo-9,10-dihydroanthracen-2-yl)methylene]-1,3-thiazolidine-2,4-dione;
(5-[(2,2-difluoro-1,3-benzodioxol-5-yl)methylene]-1,3-thiazolidine-2,4-dione;
(5Z)-5-(1,3-dihydro-2-benzofuran-5-ylmethylene)-1,3-thiazolidine-2,4-dione;
5-(1-benzofuran-5-ylmethylene)-1,3-thiazolidine-2,4-dione;
5-[(4-methyl-3-oxo-3,4-dihydro-2H-1,4-benzoxazin-6-yl)methylene]-1,3-thiazolidine-2,4-dione;
5-(1,3-benzodioxol-5-ylmethylene)-2-imino-1,3-thiazolidin-4-one;
5-Quinolin-6-ylmethylene-thiazolidine-2,4-dione;
5-Quinolin-6-ylmethylene-2-thioxo-thiazolidin-4-one;
2-Imino-5-quinolin-6-ylmethylene-thiazolidin-4-one;
5-(3-Methyl-benzo[d]isoxazol-5-ylmethylene)-thiazolidine-2,4-dione;
5-(4-Phenyl-quinazolin-6-ylmethylene)-thiazolidine-2,4-dione;
5-(4-Dimethylamino-quinazolin-6-ylmethylene)-thiazolidine-2,4-dione;
5-[(4-aminoquinazolin-6-yl)methylene]-1,3-thiazolidine-2,4-dione;
5-[(4-piperidin-1-ylquinazolin-6-yl)methylene]-1,3-thiazolidine-2,4-dione;
5-[(4-morpholin-4-ylquinazolin-6-yl)methylene]-1,3-thiazolidine-2,4-dione;
5-{[4-(benzylamino)quinazolin-6-yl]methylene}-1,3-thiazolidine-2,4-dione;
5-{[4-(diethylamino)quinazolin-6-yl]methylene}-1,3-thiazolidine-2,4-dione;
5-({4-[(pyridin-2-ylmethyl)amino]quinazolin-6-yl}methylene)-1,3-thiazolidine-2,4-dione;
5-({4-[(pyridin-3-ylmethyl)amino]quinazolin-6-yl}methylene)-1,3-thiazolidine-2,4-dione;
ethyl 1-{6-[(2,4-dioxo-1,3-thiazolidin-5-ylidene)methyl]quinazolin-4-yl}piperidine-3-carboxylate;
ethyl 1-{6-[(2,4-dioxo-1,3-thiazolidin-5-ylidene)methyl]quinazolin-4-yl}piperidine-4-carboxylate;
tert-butyl 1-{6-[(2,4-dioxo-1,3-thiazolidin-5-ylidene)methyl]quinazolin-4-yl}-L-prolinate;
5-{[4-(4-methylpiperazin-1-yl)quinazolin-6-yl]methylene}-1,3-thiazolidine-2,4-dione;
5-{[4-(4-pyrimidin-2-ylpiperazin-1-yl)quinazolin-6-yl]methylene}-1,3-thiazolidine-2,4-dione;
5-({4-[4-(4-fluorophenyl)piperidin-1-yl]quinazolin-6-yl}methylene)-1,3-thiazolidine-2,4-dione;
5-{[4-(4-benzylpiperidin-1-yl)quinazolin-6-yl]methylene}-1,3-thiazolidine-2,4-dione;
5-({4-[4-(2-phenylethyl)piperidin-1-yl]quinazolin-6-yl}methylene)-1,3-thiazolidine-2,4-dione;
5-{[4-(4-methylpiperidin-1-yl)quinazolin-6-yl]methylene}-1,3-thiazolidine-2,4-dione;
5-{[4-(4-hydroxypiperidin-1-yl)quinazolin-6-yl]methylene}-1,3-thiazolidine-2,4-dione;
1-[6-(2,4-Dioxo-thiazolidin-5-ylidenemethyl)-quinazolin-4-yl]-piperidine-4-carboxylic acid;
1-[6-(2,4-Dioxo-thiazolidin-5-ylidenemethyl)-quinazolin-4-yl]-piperidine-3-carboxylic acid;
1-[6-(2,4-Dioxo-thiazolidin-5-ylidenemethyl)-quinazolin-4-yl]-pyrrolidine-2-carboxylic acid;
5-(4-Methylamino-quinazolin-6-ylmethylene)-thiazolidine-2,4-dione;
5-(4-Methoxy-quinazolin-6-ylmethylene)-thiazolidine-2,4-dione;
2-Imino-5-(4-methylamino-quinazolin-6-ylmethylene)-thiazolidin-4-one;
2-Imino-5-((4-(piperidin-1-yl)quinazolin-6-yl)methylene)-thiazolidin-4-one;
2-Imino-5-(4-dimethylamino-quinazolin-6-ylmethylene)-thiazolidin-4-one;
5-(2-Methyl-2H-benzotriazol-5-ylmethylene)-thiazolidine-2,4-dione;
5-(3-Methyl-3H-benzotriazol-5-ylmethylene)-thiazolidine-2,4-dione;
5-(3-Ethyl-3H-benzoimidazol-5-ylmethylene)-thiazolidine-2,4-dione;
5-{[1-(4-phenylbutyl)-1H-benzimidazol-6-yl]methylene}-1,3-thiazolidine-2,4-dione;
5-[(1-prop-2-yn-1-yl-1H-benzimidazol-6-yl)methylene]-1,3-thiazolidine-2,4-dione;
5-[(1-{2-[4-(trifluoromethyl)phenyl]ethyl}-1H-benzimidazol-6-yl)methylene]-1,3-thiazolidine-2,4-dione;
5-({1-[2-(4-hydroxyphenyl)ethyl]-1H-benzimidazol-6-yl}methylene)-1,3-thiazolidine-2,4-dione;
methyl 4-{6-[(2,4-dioxo-1,3-thiazolidin-5-ylidene)methyl]-1H-benzimidazol-1-yl}cyclohexanecarboxylate;
5-({1-[2-(5-methoxy-1H-indol-3-yl)ethyl]-1H-benzimidazol-6-yl}methylene)-1,3-thiazolidine-2,4-dione;
5-({1-[(1-methyl-1H-pyrazol-4-yl)methyl]-1H-benzimidazol-6-yl}methylene)-1,3-thiazolidine-2,4-dione;
5-({1-[2-(3,4-dimethoxyphenyl)ethyl]-1H-benzimidazol-6-yl}methylene)-1,3-thiazolidine-2,4-dione;
5-({1-[2-(4-phenoxyphenyl)ethyl]-1H-benzimidazol-6-yl}methylene)-1,3-thiazolidine-2,4-dione;
5-({1-[4-(trifluoromethyl)benzyl]-1H-benzimidazol-6-yl}methylene)-1,3-thiazolidine-2,4-dione;
4-{6-[(2,4-dioxo-1,3-thiazolidin-5-ylidene)methyl]-1H-benzimidazol-1-yl}cyclohexanecarboxylic acid;
5-[(1-isobutyl-1H-benzimidazol-6-yl)methylene]-1,3-thiazolidine-2,4-dione;
5-({1-[2-(1,3-benzodioxol-4-yl)ethyl]-1H-benzimidazol-6-yl}methylene)-1,3-thiazolidine-2,4-dione;
5-({1-[2-(2-phenoxyphenyl)ethyl]-1H-benzimidazol-6-yl}methylene)-1,3-thiazolidine-2,4-dione;
5-{[1-(3,3-diphenylpropyl)-1H-benzimidazol-6-yl]methylene}-1,3-thiazolidine-2,4-dione;
5-{[1-(2-methoxybenzyl)-1H-benzimidazol-6-yl]methylene}-1,3-thiazolidine-2,4-dione;
5-{[1-(3-furylmethyl)-1H-benzimidazol-6-yl]methylene}-1,3-thiazolidine-2,4-dione;
5-[(1-propyl-1H-benzimidazol-6-yl)methylene]-1,3-thiazolidine-2,4-dione;
5-Quinoxalin-6-ylmethylene-thiazolidine-2,4-dione;
5-Quinoxalin-6-ylmethylene-2-thioxo-thiazolidin-4-one;
2-Imino-5-quinoxalin-6-ylmethylene-thiazolidin-4-one;
5-Benzothiazol-6-ylmethylene-thiazolidine-2,4-dione;
5-(3-Methyl-benzofuran-5-ylmethylene)-thiazolidine-2,4-dione;
5-(2-Bromo-3-methyl-benzofuran-5-ylmethylene)-thiazolidine-2,4-dione;
5-(3-bromo-benzofuran-5-ylmethylene)-thiazolidine-2,4-dione;
3-[5-(2,4-Dioxo-thiazolidin-5-ylidenemethyl)-benzofuran-3-yl]-acrylic acid ethyl ester;
3-[5-(2,4-Dioxo-thiazolidin-5-ylidenemethyl)-benzofuran-3-yl]-acrylic acid;
5-[3-(3-Oxo-3-piperidin-1-yl-propenyl)-benzofuran-5-ylmethylene]-thiazolidine-2,4-dione;
Methyl 1-(3-{5-[(2,4-dioxo-1,3-thiazolidin-5-ylidene)methyl]-1-benzofuran-3-yl}prop-2-enoyl)prolinate;
Methyl 1-(3-{5-[(2,4-dioxo-1,3-thiazolidin-5-ylidene)methyl]-1-benzofuran-3-yl}prop-2-enoyl)-D-prolinate;
5-({3-[3-oxo-3-pyrrolidin-1-ylprop-1-en-1-yl]-1-benzofuran-5-yl}methylene)-1,3-thiazolidine-2,4-dione;
5-({3-[3-morpholin-4-yl-3-oxoprop-1-en-1-yl]-1-benzofuran-5-yl}methylene)-1,3-thiazolidine-2,4-dione;
Methyl 1-(3-{5-[(2,4-dioxo-1,3-thiazolidin-5-ylidene)methyl]-1-benzofuran-3-yl}prop-2-enoyl)-L-prolinate;
N-cyclohexyl-3-{5-[(2,4-dioxo-1,3-thiazolidin-5-ylidene)methyl]-1-benzofuran-3-yl}-N-methylacrylamide;
3-{5-[(2,4-dioxo-1,3-thiazolidin-5-ylidene)methyl]-1-benzofuran-3-yl}-N-ethyl-N-(2-hydroxyethyl)acrylamide;
N-cyclobutyl-3-{5-[(2,4-dioxo-1,3-thiazolidin-5-ylidene)methyl]-1-benzofuran-3-yl}acrylamide;
5-({3-[3-azetidin-1-yl-3-oxoprop-1-en-1-yl]-1-benzofuran-5-yl}methylene)-1,3-thiazolidine-2,4-dione;
5-({3-[3-(1,3-dihydro-2H-isoindol-2-yl)-3-oxoprop-1-en-1-yl]-1-benzofuran-5-yl}methylene)-1,3-thiazolidine-2,4-dione;
5-({3-[3-azepan-1-yl-3-oxoprop-1-en-1-yl]-1-benzofuran-5-yl}methylene)-1,3-thiazolidine-2,4-dione;
3-{5-[(2,4-dioxo-1,3-thiazolidin-5-ylidene)methyl]-1-benzofuran-3-yl}-N-piperidin-1-ylacrylamide;
3-{5-[(2,4-dioxo-1,3-thiazolidin-5-ylidene)methyl]-1-benzofuran-3-yl}-N-(pyridin-3-ylmethyl)acrylamide;
N-cyclohexyl-3-{5-[(2,4-dioxo-1,3-thiazolidin-5-ylidene)methyl]-1-benzofuran-3-yl}acrylamide;
5-({3-[3-(4-methylpiperazin-1-yl)-3-oxoprop-1-en-1-yl]-1-benzofuran-5-yl}methylene)-1,3-thiazolidine-2,4-dione;
N-cycloheptyl-3-{5-[(2,4-dioxo-1,3-thiazolidin-5-ylidene)methyl]-1-benzofuran-3-yl}acrylamide;
5-({3-[3-(2,5-dihydro-1H-pyrrol-1-yl)-3-oxoprop-1-en-1-yl]-1-benzofuran-5-yl}methylene)-1,3-thiazolidine-2,4-dione;
N-cyclopentyl-3-{5-[(2,4-dioxo-1,3-thiazolidin-5-ylidene)methyl]-1-benzofuran-3-yl}acrylamide;
3-[5-(2,4-Dioxo-thiazolidin-5-ylidenemethyl)-benzofuran-3-yl]-propionic acid ethyl ester;
3-[5-(2,4-Dioxo-thiazolidin-5-ylidenemethyl)-benzofuran-3-yl]-propionic acid;
5-[3-(3-Oxo-3-piperidin-1-yl-propyl)-benzofuran-5-ylmethylene]-thiazolidine-2,4-dione;
6-(2,4-Dioxo-thiazolidin-5-ylidenemethyl)-2,3-dihydro-benzo[1,4]oxazine-4-carboxylic acid tert-butyl ester;
5-(3,4-Dihydro-2H-benzo[1,4]oxazin-6-ylmethylene)-thiazolidine-2,4-dione;
5-(4-Benzoyl-3,4-dihydro-2H-benzo[1,4]oxazin-6-ylmethylene)-thiazolidine-2,4-dione;
5-(4-Acetyl-3,4-dihydro-2H-benzo[1,4]oxazin-6-ylmethylene)-thiazolidine-2,4-dione;
6-(2,4-Dioxo-thiazolidin-5-ylidenemethyl)-benzo[1,4]oxazine-4-carboxylic acid tert-butyl ester;
[6-(2,4-Dioxo-thiazolidin-5-ylidenemethyl)-3-oxo-2,3-dihydro-benzo[1,4]-oxazin-4-yl]-acetic acid methyl ester;
N-Benzyl-2-[6-(2,4-dioxo-thiazolidin-5-ylidenemethyl)-3-oxo-2,3-dihydro-benzo[1,4]oxazin-4-yl]-acetamide;
5-(4-Butyl-3-oxo-3,4-dihydro-2H-benzo[1,4]oxazin-6-ylmethylene)-thiazolidine-2,4-dione;
5-(4-Benzyl-3-oxo-3,4-dihydro-2H-benzo[1,4]oxazin-6-ylmethylene)-thiazolidine-2,4-dione;
5-(2-Chloro-benzofuran-5-ylmethylene)-thiazolidine-2,4-dione;
5-(3-Amino-benzo[d]isoxazol-5-ylmethylene)-thiazolidine-2,4-dione;
5-(3-Phenylethynyl-benzofuran-5-ylmethylene)-thiazolidine-2,4-dione;
5-Benzo[1,2,5]thiadiazol-5-ylmethylene-thiazolidine-2,4-dione;
5-Benzo[1,2,5]oxadiazol-5-ylmethylene-thiazolidine-2,4-dione;
5-(2-Methyl-benzofuran-6-ylmethylene)-thiazolidine-2,4-dione;
5-(2-Carboxymethyl-benzofuran-6-ylmethylene)-thiazolidine-2,4-dione;
5-(3-Bromo-2-fluoro-2,3-dihydro-benzofuran-6-ylmethylene)-thiazolidine-2,4-dione;
5-(2-Fluoro-benzofuran-6-ylmethylene)-thiazolidine-2,4-dione;
N-(5-Benzo[1,3]dioxol-5-ylmethylene-4-oxo-thiazolidin-2-ylidene)-2-chlorobenzenesulfonamide;
Ethanesulfonic acid (5-benzo[1,3]dioxol-5-ylmethylene-4-oxo-thiazolidin-2-ylidene)-amide;
N-(5-Benzo[1,3]dioxol-5-ylmethylene-4-oxo-thiazolidin-2-ylidene)-3-chlorobenzenesulfonamide;
5-Chloro-1,3-dimethyl-1H-pyrazole-4-sulfonic acid (5-benzo[1,3]dioxol-5-ylmethylene-4-oxo-thiazolidin-2-ylidene)amide;
3-(5-Benzo[1,3]dioxol-5-ylmethylene-4-oxo-thiazolidin-2-ylidenesulfamoyl)-thiophene-2-carboxylic acid methyl ester;
6-Chloro-pyridine-3-sulfonic acid (5-benzo[1,3]dioxol-5-ylmethylene-4-oxo-thiazolidin-2-ylidene)amide;
Quinoline-8-sulfonic acid (5-benzo[1,3]dioxol-5-ylmethylene-4-oxo-thiazolidin-2-ylidene)amide;
N-(5-Benzo[1,3]dioxol-5-ylmethylene-4-oxo-thiazolidin-2-ylidene)-benzenesulfonamide;
N-(5-Benzo[1,3]dioxol-5-ylmethylene-4-oxo-thiazolidin-2-ylidene)-4-methylbenzenesulfonamide;
N-(5-Benzo[1,3]dioxol-5-ylmethylene-4-oxo-thiazolidin-2-ylidene)-methanesulfonamide;
N-[5-(2,2-Difluoro-benzo[1,3]dioxol-5-ylmethylene)-4-oxo-thiazolidin-2-ylidene]-benzenesulfonamide;
N-[5-(2,2-Difluoro-benzo[1,3]dioxol-5-ylmethylene)-4-oxo-thiazolidin-2-ylidene]-4-methyl-benzenesulfonamide;
N-[5-(2,2-Difluoro-benzo[1,3]dioxol-5-ylmethylene)-4-oxo-thiazolidin-2-ylidene]-methanesulfonamide;
Biphenyl-2-sulfonic acid (5-benzo[1,3]dioxol-5-ylmethylene-4-oxo-thiazolidin-2-ylidene)amide;
Pyridine-3-sulfonic acid (5-benzo[1,3]dioxol-5-ylmethylene-4-oxo-thiazolidin-2-ylidene)amide;
3-(4-Oxo-5-quinolin-6-ylmethylene-thiazolidin-2-ylidenesulfamoyl)-thiophene-2-carboxylic acid methyl ester;
2-Chloro-N-(4-oxo-5-quinolin-6-ylmethylene-thiazolidin-2-ylidene)-benzenesulfonamide;
3-(5-Benzo[1,3]dioxol-5-ylmethylene-4-oxo-thiazolidin-2-ylidenesulfamoyl)-thiophene-2-carboxylic acid;
5-Benzo[1,3]dioxol-5-ylmethylene-4-oxo-thiazolidin-2-ylidene-cyanamide;
5-Benzo[1,3]dioxol-5-ylmethylene-thiazolidine-2,4-dione 2-(O-methyl-oxime);
4-Oxo-5-quinoxalin-6-ylmethylene-thiazolidin-2-ylidene-cyanamide;
5-Benzo[1,3]dioxol-5-ylmethylene-2-benzylimino-thiazolidin-4-one;
2-Benzylimino-5-quinolin-6-ylmethylene-thiazolidin-4-one;
2-Propylimino-5-quinolin-6-ylmethylene-thiazolidin-4-one;
5-Benzo[1,3]dioxol-5-ylmethylene-2-propylimino-thiazolidin-4-one; and
5-(4-Dimethylamino-quinazolin-6-ylmethylene)-2-methylamino-thiazol-4-one;
or a pharmaceutically acceptable salt thereof.

35. A composition comprising an erythropoetin (EPO), a variant, or an analogue thereof, a PI3 Kinase gamma inhibitor according to claim 19 and a pharmaceutically acceptable excipient.

36. A composition according to claim 35 which is an injectable liquid.
